(19) United States
(12) Patent Application Publication
(10) Pub. No.: US 2023/0390452 A1
(43) Pub. Date: Dec. 7, 2023

(54) PHOTOCURABLE LIQUID ADHESIVES, AND KITS AND METHODS FOR SAME

(71) Applicant: Cook Biotech Incorporated, West Lafayette, IN (US)

(72) Inventor: Sydney E. Hollingshead, Lafayette, IN (US)

(73) Assignee: Cook Biotech Incorporated, West Lafayette, IN (US)

(21) Appl. No.: 18/313,049

(22) Filed: May 5, 2023

Related U.S. Application Data
(60) Provisional application No. 63/338,656, filed on May 5, 2022.

(51) Int. Cl. A61L 24/10; A61M 5/19; A61B 17/00

(52) U.S. Cl. CPC A61L 24/104; A61B 2017/00495; A61B 17/00491; A61M 5/19

(57) ABSTRACT

Described are photocurable liquid adhesives, kits for preparing photocurable liquid adhesives, and methods for their preparation and use. Kits can include first and second components that can be mixed to prepare a photocurable liquid adhesive. The first component can include a liquid preparation including an aqueous liquid, one or more polymers containing phenolic groups, and a photoactivatable metal ligand complex. The second component can include an electron acceptor. Mixing the first component and the second component can create a photocurable liquid adhesive. The first and second components can be packaged in containers such as syringes, and the kits can include the containers and a cannulated coupler for fluidly coupling the containers to mix the container contents to prepare the photocurable liquid adhesive. A syringe dispensing tip and/or a light source for curing the photocurable liquid adhesive can also be included in the kits. Advantageous photocurable liquid adhesives are described that, absent curing, remain in flowable liquid form during typical temperatures of storage and use. The adhesives can include in or as a phenolic group-containing polymer component one or more of collagen, phenol enriched collagen, gelatin, phenol enriched gelatin, a collagen peptide composition, or a phenol enriched collagen peptide composition.

### CROSS REFERENCE TO RELATED APPLICATIONS

This application claims the benefit of U.S. Provisional Application No. 63/338,656 filed May 5, 2022 entitled PHOTOCURABLE LIQUID ADHESIVES, AND KITS AND METHODS FOR SAME, which is hereby incorporated by reference.

### BACKGROUND

In certain aspects, the present disclosure relates to photocurable liquid adhesives that include a polymer containing phenolic groups, a photoactivatable metal ligand complex, and an electron acceptor, and to kits and methods for their preparation and use.

As further background, tissue adhesives have been suggested and used as alternatives in surgical procedures to mechanical means of connecting tissues such as sutures and staples. As exemplary uses, tissue adhesives can hold cut or separated areas of tissue together to allow healing, serve as a barrier to leakage, provide local delivery of exogenous substances, and/or provide hemostasis. A number of tissue adhesives are known, including as examples fibrin glues, albumin-glutaraldehyde based compounds, cyanoacrylates, polyethylene glycol polymers, or collagen-based adhesives. Tissue adhesives may also be utilized in the manufacture of implants to treat various medical conditions in patients, and in other areas.

In light of the background work in this area, there remain needs for improved and/or alternative adhesives as well as kits and methods for their preparation and use.

### SUMMARY

In one aspect, provided is a kit for preparing a photocurable liquid adhesive. The kit includes a first container defining a first chamber and containing a sterile liquid preparation in the first chamber, the sterile liquid preparation including an aqueous liquid, one or more polymers containing phenolic groups, and a metal ligand complex. The kit further includes a second container defining a second chamber and containing a sterile electron acceptor in the second chamber. The liquid preparation and the electron acceptor can be mixed to prepare a photocurable liquid adhesive effective to form a diphenolic crosslinked polymer hydrogel when photocured.

In another aspect, provided is a liquid adhesive composition. The composition includes a photocurable liquid preparation includes an aqueous liquid and one or more polymers containing phenolic groups selected from collagen, phenol enriched collagen, gelatin, phenol enriched gelatin, a collagen peptide composition having an average molecular weight of less than 20 kilodaltons, and a phenol enriched collagen peptide composition having an average molecular weight of less than 20 kilodaltons. The photocurable liquid preparation further includes a photoactivatable metal ligand complex, and an electron acceptor. Preferably the photocurable liquid preparation, absent curing, can remain in a liquid state at 20° C. In preferred forms the photocurable liquid preparation, absent curing, remains in a liquid state throughout the temperature range of 20° C. to 37° C. In another aspect, provided is a kit for preparing such a photocurable liquid adhesive. The kit includes a first container defining a first chamber and containing a sterile liquid preparation in the first chamber, the sterile liquid preparation including the aqueous liquid, the one or more polymers, and the photoactivatable metal ligand complex. The sterile liquid preparation remains in a liquid state at 20° C. and in preferred forms remains in a liquid state throughout the temperature range of 20° C. to 37° C. The kit further includes a second container defining a second chamber and containing a sterile electron acceptor in the second chamber. The sterile liquid preparation and the sterile electron acceptor are mixable to prepare the photocurable liquid adhesive. In some forms, the one or more polymers includes or is constituted of gelatin and/or phenol enriched gelatin, and the liquid preparation also includes an agent that inhibits thermoreversible gelling of the gelatin and/or phenol enriched gelatin, preferably wherein the agent is urea. In other forms, the one or more polymers includes or is constituted of the collagen peptide composition and/or the phenol enriched collagen peptide composition, and the collagen peptide composition exhibits no thermoreversible gelation activity.

Additional aspects herein relate to methods for preparation of, or methods for use of, photocurable adhesive compositions. These methods can involve the use of kits or photocurable adhesives as disclosed above and/or elsewhere herein.

Still further aspects and embodiments, and features and advantages thereof, will be apparent to those skilled in the pertinent field from the disclosures herein.

### BRIEF DESCRIPTION OF THE FIGURES

FIG. 1 provides a schematic view of one embodiment of a kit for preparing a photocurable medical adhesive.

FIG. 2 provides a graph illustrating the extent of phenol enrichment of gelatin using varied concentrations of Bolton-Hunter reagent, as further described in Example 5 below.

### DETAILED DESCRIPTION

Reference will now be made to certain embodiments, some of which are illustrated in the drawings, and specific language will be used to describe the same. It will nevertheless be understood that no limitation of the scope of the disclosure herein is thereby intended. Any alterations and further modifications in the described embodiments, and any further applications of the principles as described herein, are contemplated as would normally occur to a person skilled in the art to which the embodiments relate.

As disclosed above, aspects of the present disclosure relate to kits for preparing photocurable medical adhesives, to photocurable medical adhesives, and to methods of preparation and use of such kits and adhesives.

In certain preferred forms, the photocurable adhesive is comprised of an aqueous liquid in which are dissolved one or more polymers containing phenolic groups, a photoactivatable metal ligand complex, and an electron acceptor. The polymer(s) containing phenolic groups can be natural polymer(s) (for example a protein) or synthetic polymer(s). Suitable phenolic group-containing proteins for use in the photocurable adhesive include, for example, fibrinogen, fibrin, collagen, keratin, gelatin, fibronectin, serum albumin, elastin, beta-lactoglobulin, glycinin, glutens, gliadins, resilin and/or laminin, or admixtures thereof. Such proteins or other natural polymers may be derived from human or animal sources or can be synthetically produced for instance using recombinant techniques. In some forms, the protein may be denatured to encourage the formation of diphenolic covalent crosslinks upon photocuring and/or may be a phenol enriched protein (e.g. modified to increase the number of 4-hydroxyphenyl groups). Denaturation of a protein may be accomplished by raising or lowering the pH of a solution containing the matrix protein, decreasing or increasing the ionic strength of a solution containing the matrix protein, hydrolysis, or in other ways known to a person skilled in the art. Chemical modification to form a phenol enriched polymer material, e.g. including chemically added 4-hydroxyphenyl propionyl groups (which are much like tyrosine groups), may be achieved by any suitable method. In some forms, such chemical modification may include the modification of amino acid side chains of a protein to include moieties that contain a phenolic hydroxyl group such as a 4-hydroxyphenyl group (e.g. as in tyrosine and similar groups). By way of example primary amines such as the lysine residues and/or terminal amine groups in a protein may be modified to add 4-hydroxyphenyl propionyl groups using the known Bolton-Hunter reagent (N-succinimidyl-3-[4-hydroxyphenyl]propionate) or the known water soluble Bolton-Hunter reagent (sulfosuccinimidyl-3-[4-hydroxyphenyl]propionate). In certain forms, the photocurable adhesive will include a mixture of an amount of a protein (especially collagen, gelatin or a collagen peptide composition as described herein) with an amount of the corresponding phenol enriched protein. For example, the photocurable adhesive may include a combination of the parent (unmodified) protein and the corresponding phenol enriched protein in a dry weight ratio in the range of about 1:10 to about 10:1, or in the range of about 1:5 to about 5:1, and in some particular forms in a dry weight ratio of about 5:1, about 1:1, or about 1:5. In certain embodiments, such ranges of ratios or such ratios are used when the parent (unmodified) protein is collagen, gelatin, or a collagen peptide composition.

While not wishing to be bound by theory, it is believed that the curing mechanism involves irradiation of the metal ligand complex to induce an excited state, followed by transfer of an electron from the metal to an electron acceptor. The oxidized metal then extracts an electron from a phenolic group-containing side chain such as a tyrosine side chain in the protein or other polymer to produce a radical that reacts with a nearby tyrosine or other phenolic group to form a dityrosine or other diphenolic bond. A direct cross-link (without any bridging moiety) is created quickly in this photo-initiated chemical reaction.

The term “photoactivatable metal ligand complex” as used herein means a metal ligand complex in which the metal can enter an excited state when irradiated such that it can donate an electron to an electron acceptor in order to move to a higher oxidation state and thereafter extract an electron from a side chain of an amino acid residue of a matrix protein to produce a free radical without reliance upon the formation of singlet oxygen. Suitable metals include but are not limited to Ru(II), Pd(II), Cu(II), Ni(II), Mn(II) and Fe(III) in the form of a complex which can absorb light in the visible region, for example, a Ru(II) bipyridyl complex, a Pd(II) porphyrin complex, a sulfonatophenyl Mn(II) complex or a Fe(III) protoporphyrin complex, more particularly, a Ru(II) bispyridyl complex or a Pd(II) porphyrin, in particular, a Ru(II) (bpy)3 complex (i.e. a tris(2,2′-bipyridine)ruthenium(II) complex) such as [Ru(II) (bpy)3] Cl2 (i.e. tris(2,2′-bipyridyl) ruthenium (II) chloride). Efficient cross-linking occurs in the presence of an electron acceptor, and requires only moderate intensity visible (e.g. white) light.

As used herein the term “electron acceptor” refers to a chemical entity that accepts electron transferred to it and so refers to an easily reduced molecule (or oxidizing agent) with a redox potential sufficiently positive to facilitate the cross-linking reaction. A range of electron acceptors will be suitable. In an embodiment, the electron acceptor is a peracid, a cobalt complex, a cerium (IV) complex, or an organic acid. Typically the electron acceptor is a persulfate, periodate, perbromate or perchlorate compound, vitamin B12, Co(III) (NH3)SCI2+, cerium (IV) sulphate dehydrate, ammonium cerium (IV) nitrate, oxalic acid or EDTA. Preferably, the persulfate anion is used as the electron acceptor. The standard oxidation-reduction potential for the reaction:

S₂O₈²⁻+2H⁺+2e^{˜}→2HSO₄⁻

is 2.1 V, as compared to 1.8 V for hydrogen peroxide (H₂O₂). This potential is higher than the redox potential for the permanganate anion (MnO₄⁻) at 1.7 V, but slightly lower than that of ozone at 2.2 V.

The term “phenolic group” as used herein means a phenyl group having a hydroxyl group attached directly to a carbon atom of the phenyl ring. A phenolic group can include other functional groups attached to other carbon atoms of the phenyl ring, or can be free of functional groups attached to the other carbon atoms of the phenyl ring (i.e. can include a -Phe-OH group where Phe represents a phenyl ring in which hydrogen groups (—H) occupy the remaining carbons of the ring). Preferred phenolic groups are 4-hydroxyphenyl groups, as for example occurs in a tyrosine residue of a protein or in a 4-hydroxyphenyl propionyl group.

The term “phenol enriched” as applied to a polymer substance herein (e.g. collagen, gelatin, or a collagen peptide composition) means that the polymer material has been chemically modified to increase the number of phenolic groups in the polymer material. Thus, “phenol enriched collagen” refers to collagen that has been chemically modified to increase the number of phenolic groups in the collagen, “phenol enriched gelatin” refers to gelatin that has been chemically modified to increase the number of phenolic groups in the gelatin, and “phenol enriched collagen peptide composition” refers to a collagen peptide composition that has been chemically modified to increase the number of phenolic groups in the collagen peptide composition. In certain forms, the phenolic groups can be 4-hydroxylphenyl groups, for example as present in 4-hydroxyphenyl propionyl groups, which can be added for example using a known Bolton-Hunter reagent. In some aspects, the phenol enriched polymer material (e.g. collagen, gelatin, or collagen peptide composition) will have a Phe/P value of at least about 7, and in certain forms in the range of about 7 to about 35, or in the range of about 15 to about 35, or in the range of about 18 to about 25, where the Phe/P value is the number of moles of phenolic groups per mole of polymer in the polymer material. The Phe/P value for a polymer material can be determined using standard techniques therefor, including for example using an absorbance assay at a wavelength of 280 nm. Moderate Phe/P ranges for the phenol enriched polymer, as recited above, are preferred in some aspects, as modification to higher Phe/P values has been found to decrease the solubility of the material in aqueous media (see e.g. Example 5 below for phenol enriched gelatin).

In preferred forms, a multi-component system is provided for preparing a photocurable adhesive as described above. A first component can include water, the polymer(s) containing phenolic groups and the metal ligand complex; and, a second component can include the electron acceptor. The second component can be in the form a dry powder or in the form of a flowable liquid, for example a flowable liquid including an aqueous liquid and the electron acceptor. The first and second components can be mixed to form a flowable photocurable adhesive that, when exposed to visible light, cures by the formation of covalent diphenolic crosslinks between molecules of the polymer(s).

Certain embodiments herein provide a kit for preparing a photocurable adhesive. The kit can include a first container, for example a syringe, a syringe barrel or a vial, defining a first chamber and containing a sterile liquid preparation in the first chamber. The sterile liquid preparation includes an aqueous liquid (such as water or a buffered aqueous liquid), the phenolic polymer(s) preferably dissolved in the aqueous liquid, and a photoactivatable metal ligand complex. The kit can further include a second container, for example a syringe, a syringe barrel or a vial, defining a second chamber and containing an electron acceptor in the second chamber. The sterile liquid preparation and the electron acceptor are mixable to prepare a photocurable liquid adhesive effective to form a diphenolic crosslinked polymer hydrogel when photocured. In some forms, the kit can also include a cannulated connector for fluidly connecting the first chamber and the second chamber and/or a visible light source (e.g. a battery-powered light emitting diode visible light source) for curing the photocurable adhesive. Other embodiments herein relate to products that include the first container having the sterile liquid preparation in the first chamber, including variants of the first container and sterile liquid preparation as described herein. Such first container products are for example usable in kits, e.g. including second containers containing the electron acceptor and/or one or more other kit components or features as disclosed herein, and/or usable in related methods of preparing photocurable liquid adhesive compositions.

In some forms, the sterile liquid preparation in the first chamber includes collagen, phenol enriched collagen, gelatin, phenol enriched gelatin, a collagen peptide composition, or a phenol enriched collagen peptide composition. These polymer materials can be used either singly or in combination. For example, the photocurable adhesive may include a combination of collagen and phenol enriched collagen, a combination of gelatin and phenol enriched gelatin, or a combination of a collagen peptide composition and a phenol enriched collagen peptide composition. In each case, the dry weight ratio of the parent polymeric material and its corresponding phenol enriched polymeric material can be in the range of about 1:10 to about 10:1, or about 1:5 to about 5:1, or in some particular forms about 1:5, about 1:1, or about 2:1, or about 5:1. Mixtures of two or more of collagen, gelatin, and a collagen peptide composition (each in its native form without phenol enrichment or as a phenol enriched polymeric material) can also be used.

In addition or alternatively, the sterile liquid preparation that includes collagen, phenol enriched collagen, gelatin, phenol enriched gelatin, a collagen peptide composition, or a phenol enriched collagen peptide composition, or any mixture of two or more thereof, can exhibit the property of not thermoreversibly gelling when cooled to 20° C., for example exhibiting no thermoreversible gelation activity or having a thermoreversible gelation temperature below 20° C., or below 15° C. In some forms, the sterile liquid preparation comprises gelatin, phenol enriched gelatin, or a mixture thereof, and the liquid preparation also includes a biocompatible agent that inhibits the thermoreversible gelling of the gelatin (when present) and/or of the phenol enriched gelatin (when present). Urea is a preferred biocompatible agent that inhibits this thermoreversible gelling, and can be used for example at a concentration in the range of about 1 molar to 5 molar in the liquid preparation, more typically about 3 molar to about 4.5 molar, and in some forms about 3.8 molar to about 4.5 molar. In other forms, the sterile liquid preparation includes a collagen peptide composition (sometimes also referred to as a “collagen hydrolysate”—where the collagen molecules have been cleaved to molecular weights below that of thermoreversible gelatin) and/or a phenol enriched collagen peptide composition, that has an average molecular weight (M w) below about 20,000 kilodaltons, more preferably below about 15,000 kilodaltons, and typically in the range of about 2,000 to about 12,000 kilodaltons. It will be understood that reference to the average molecular weight of a phenol enriched collagen peptide composition or another phenol enriched polymer herein refers to the average molecular weight after phenol enrichment (which can of course slightly increase molecular weights). At these relatively low molecular weights, the collagen peptide composition and/or the phenol enriched collagen peptide composition can exhibit no thermoreversible gelation activity when dissolved in an aqueous liquid and held at 20° C. (or in some typical forms can exhibit no thermoreversible gelling activity at any temperature when dissolved in an aqueous liquid). This can allow the liquid preparation to remain a liquid when cooled to a temperature of 20° C., or to a temperature of 15° C. It will be understood that the liquid preparation may also remain a liquid at temperatures above and below these specified temperatures, and may remain a liquid throughout a temperature range expected to encompass room temperature storage and normal use temperatures, for example in the range of about 20° C. to about 37° C.

The sterile liquid preparation can include the polymer(s) containing phenolic groups in any suitable concentration. In some forms, the total concentration of the polymer(s) present in the sterile liquid preparation will be in the range of about 1% to about 40% weight/volume, more typically about 10% to about 40% weight/volume. In certain preferred forms, the sterile liquid preparation will include collagen, phenol enriched collagen, gelatin, phenol enriched gelatin, a collagen peptide composition, a phenol enriched collagen peptide composition, or any combination thereof, at a concentration in the range of about 20% to about 35% weight/volume, or in the range of about 25% to about 35% weight/volume. In some forms, the sterile liquid preparation, and/or photocurable liquid adhesives prepared using it, can be a flowable viscous liquid, for example having a viscosity at 20° C. of greater than about 300 centipoise, or greater than about 500 centipoise, and typically in the range of about 500 to about 20000 centipoise or in the range of about 1000 to about 10000 centipoise.

In some forms the sterile liquid preparation and/or photocurable liquid adhesive can be free from viscosity-increasing polymers other than the one or more phenolic group containing polymers, and in other forms the sterile liquid preparation and/or photocurable liquid adhesive can contain one or more viscosity-increasing polymers other than the phenol group containing polymer(s). In some embodiments, the one or more phenolic group containing polymers will include or will be constituted of phenolic group containing polymer(s) having an average molecular weight (M w) in the range of about 40 kilodaltons to about 250 kilodaltons, or in the range of about 60 kilodaltons to about 200 kilodaltons, or in the range of about 80 kilodaltons to about 150 kilodaltons. These ranges can, for example, apply to gelatin or phenol enriched gelatin when used as a phenolic group containing polymer. Such moderate to high molecular weight phenolic group containing polymer(s) can contribute to relatively viscous photocurable liquid adhesive compositions that more beneficially remain on a surface or in a region where they are applied and where they can be photocured, as opposed to a relatively non-viscous liquid that may rapidly flow away from the area of application before photocuring can be achieved.

The sterile liquid preparation can provide the photoactivatable metal ligand complex to the prepared photocurable adhesive in a suitable amount to catalyze the formation of covalent diphenolic crosslinks upon photocuring the photocurable adhesive to provide the covalently crosslinked hydrogel. In some forms, the sterile liquid preparation will include the metal ligand complex at concentration in the range of about 0.1 millimolar (mM) to about 5 mM. Where a Ru(II) (bpy)₃ complex such as [Ru(II) (bpy)₃] Cl₂ is used as the metal ligand complex, preferred sterile liquid preparations will include it at a concentration in the range of about 0.2 to about 2 mM, more desirably about 0.4 to about 1 mM. Where the electron acceptor to be mixed with the sterile liquid preparation is in dry powder form, the prepared photocurable liquid adhesive can have these same concentrations of the metal ligand complex. Where the electron acceptor is provided in a solution to be combined with the sterile liquid preparation, the concentration of the photoactivatable metal ligand complex in the prepared photocurable liquid adhesive can be reduced relative to that in the sterile liquid preparation. In some such forms, the volume of the sterile liquid preparation, the volume of the solution of electron acceptor, and the concentration of the photoactivatable metal ligand complex in the sterile liquid preparation, can be selected to provide a concentration of the photoactivatable metal ligand complex in the prepared photocurable liquid adhesive that is within the above-referenced concentration range values given for the sterile liquid preparation.

The sterile liquid preparation can have been terminally sterilized within the first chamber to render it sterile, for instance using sterilizing radiation applied to a package containing the first container. However, in some preferred forms the liquid preparation is sterilely prepared, for example including passage of the liquid preparation through a sterile filter, and then filled into the first chamber in a sterile filling operation. Such sterilely-filled liquid preparations in the first chamber can therefore be free from exposure to sterilizing radiation, and thus can be free from any degradation of the polymer(s) containing phenolic groups caused by the sterilizing radiation. In some forms the liquid preparation can be in a heated condition to reduce its viscosity during passage through the sterile filter. Also in some forms, the first container having the first chamber containing the sterilely-filled liquid preparation can be sealed within a sterile barrier package under sterile conditions. Further, such a sterile barrier package is preferably impermeable to visible light or any light that would photoactivate the photoactivatable metal ligand complex, as can be provided for example by a foil pouch package. In addition or alternatively, the first container can be impermeable to visible light or to light that would photoactivate the photoactivatable metal ligand complex. In the disclosed kits and products, these or other means for shielding the sterile liquid preparation from light that would photoactivate the photoactivatable metal ligand complex can be provided in desirable forms.

The electron acceptor in preferred embodiments is terminally sterilized within the second chamber, for example using ethylene oxide gas or sterilizing radiation (e.g. e-beam), but in other forms can be sterilely prepared and then loaded into the second chamber by a sterile filling operation. In certain variants, the electron acceptor is provided as a dry powder in the second chamber, for example as a dry powder of one member, or two or more members, of the group consisting of a persulfate, periodate, perbromate or perchlorate compound, vitamin B12, Co(III) (NH3)SCI2+, cerium (IV) sulphate dehydrate, ammonium cerium (IV) nitrate, oxalic acid and EDTA. Preferably, the electron acceptor is or includes a persulfate compound. In other variants, the electron acceptor can be provided in the second chamber as a solution of the electron acceptor (e.g. any of those listed above) in a solvent, preferably an aqueous solvent such as water or a mixture of water and a co-solvent. In still further forms of multi-part adhesive products and related kits, the electron acceptor can be in a dry powder form as discussed, and a third container (e.g. syringe or vial) can contain a sterile liquid carrier, typically an aqueous liquid, to be mixed with the dry powder form electron acceptor to prepare an electron acceptor solution, and such electron acceptor solution can then be mixed with the liquid preparation containing the polymer(s) including phenolic groups, the aqueous liquid, and the metal ligand complex, to prepare the photocurable adhesive composition.

In some aspects, the first container and/or the second container can be a syringe barrel, and the first chamber and/or the second chamber can be a chamber defined within the syringe barrel(s). In each case, the syringe barrel can be part of a syringe device also including a plunger arranged to apply pressure to and expel the contents of the chamber of the syringe barrel from an opening in a tip of the syringe barrel. Where the first container and the second container are syringe barrels, they can be connectable to one another and respective plungers associated with the syringe barrels can be used to drive the contents of the barrels back and forth between the barrels so as to mix the contents of the respective syringe barrels with one another. In some forms, the syringe barrels can have respective tips adapted to directly connect to one another by friction fit, by threaded attachment, or otherwise. In other forms, the syringe barrels can have respective tips that are configured for attachment to a separate cannulated device to fluidly couple the respective chambers of the syringe barrels to one another. For example, the syringe tips can each include a Luer connection feature or other threaded feature for connection to a corresponding feature on an end of the separate cannulated device. Illustratively, commercially available cannulated Luer lock connectors can be used to connect first and second syringe barrels with correspondingly mating Luer lock tips.

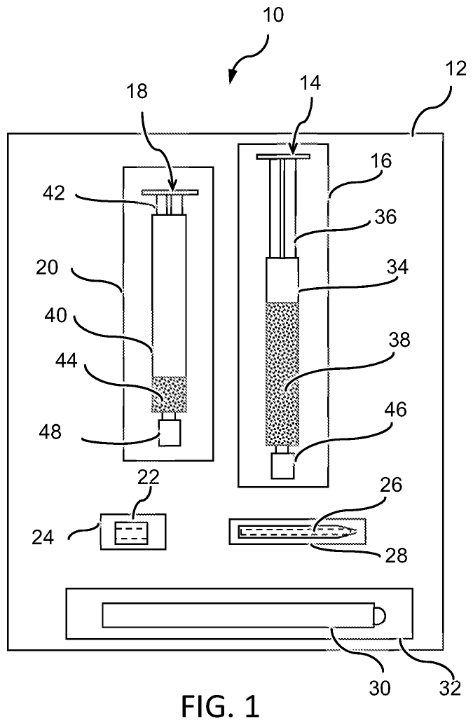

In these regards, FIG. 1 provides a schematic illustration of one embodiment of a kit 10 for preparing a photocurable adhesive herein. Kit 10 includes an outer medical package 12 such as a box or tray) containing a first syringe 14 within a package 16, a second syringe 18 within a medical package 20, a cannulated syringe coupler 22 optionally including an internal static mixer in a package 24, a syringe tip dispenser 26 within a package 28, and a light source within a package 32. First syringe 14 includes a syringe barrel 34 and a plunger 36. Syringe barrel 34 defines an inner chamber that contains a sterile liquid preparation 38 of a multi-part adhesive as discussed herein. The package 16 containing the first syringe 14 is preferably impermeable to visible light, for example as can be provided by a foil pouch. The inner packages 16, 24, 28, 32 can in some forms be double pouch packages, where an outer pouch contains an enclosed pouch that is sterile both inside and on its outer surfaces, where the enclosed pouch in turn contains the syringe 14 or 18, the syringe coupler 22, the tip dispenser 26, or the light source 30, as the case may be. The sterile liquid preparation 38 can, for example, have a volume in the range of about 0.25 ml to about 50 ml in some forms, more typically in the range of about 0.25 ml to about 20 ml, and in certain preferred forms in the range of about 0.5 ml to about 10 ml. Second syringe 18 includes a syringe barrel 40 and a plunger 42. Second syringe 18 defines an inner chamber containing a sterile electron acceptor 44 of a two-part adhesive as discussed herein. The first syringe 14 and the second syringe 18 can include respective tip caps 46 and 48. In use of the kit 10, the outer package 12 and then the inner packages 16, 24, 28, 32 can be opened to access the first and second syringes 14 and 18, the cannulated syringe coupler 22, the syringe tip dispenser 26, and the light source 30. The caps 46 and 48 can be removed from the tips of their respective syringes, and the coupler 22 used to fluidly couple the syringes 14 and 18 by connecting a tip of syringe 14 to one end of coupler 22 and the tip of syringe 18 to the other end of coupler 22. The plungers 36 and 42 can be alternately depressed to combine the liquid preparation 38 and electron acceptor 44 and to mix them together by forcing them back and forth through the cannulated coupler 22 and between the respective chambers of syringes 14 and 18. Where the electron acceptor 44 is in a dry powder form, it will be beneficial to first depress plunger 36 to drive the liquid preparation 38 through the coupler 22 and into the chamber of syringe 18 to wet and at least partially or fully dissolve the electron acceptor 44 in the liquid preparation 38, after which alternate depression of the plungers 36 and 42 can be continued to mix the materials (and beneficially fully dissolve the electron acceptor if not already fully dissolved) to prepare the photocurable liquid adhesive. After mixing is complete, the prepared photocurable adhesive can be driven into either first syringe 14 or second syringe 18, and the syringe containing the photocurable adhesive can be decoupled from the coupler 22. If desired, the syringe tip dispenser 26 can be coupled to the tip of the syringe containing the photocurable adhesive, and the adhesive dispensed through the dispenser 26 by depressing the plunger of the syringe. Alternately, the adhesive can be dispensed directly from the syringe tip or by other means, as will be understood. The dispensed photocurable adhesive (e.g. as positioned on or in a patient or on another material surface) can then be cured using the light source 30 (e.g. a battery-powered LED light source). While these discussions describe certain kit components and modes of their use, it will of course be understood that other combinations of fewer or more kit components and other modes of use are contemplated as being within the scope of the present disclosure.

The photocurable liquid adhesive can be used in a variety of patient treatments or in the manufacture of medical implants or other devices where one component is to be adhered to another. In illustrative patient treatments, the photocurable liquid adhesive can be applied to patient tissues that need to be adhered to other patient tissues or an implant material, and then photocured to cause such adherence. In other patient treatment, the photocurable liquid adhesive can be applied to patient tissues or regions where a sealant is needed, and then photocured to provide the sealant. In still other patient treatments, the photocurable liquid adhesive can be applied to patient regions where tissue bulking is needed, and then photocured to provide tissue bulking. In this regard, it has been discovered that the photocurable liquid adhesive can be effectively photocured even where it lies beneath a layer or volume of tissue of the patient, by directing visible light at and through the layer or volume of patient tissue and into a volume of the photocurable liquid adhesive residing below or behind the layer or volume of patient tissue (e.g. with the volume of photocurable liquid adhesive having been injected through a needle or other cannula extended through the layer or volume of patient tissue). Convenient and effective tissue bulking procedures are thereby achieved. In other uses, the photocurable liquid adhesive has been found to effectively bond together biocompatible material layers, for example decellularized extracellular matrix material layers, in the preparation of laminated medical implants. These and other medical uses (including human and veterinary uses) for the photocurable liquid adhesives and kits disclosed herein will be apparent to those skilled in the relevant art.

To promote further understanding of certain embodiments disclosed herein, reference will now be made to specific Examples. It will be understood that these Examples are illustrative, and not limiting, of embodiments disclosed herein.

### Example 1

### Preparation of Two-Part Adhesive Composition

First Part: A first flowable liquid composition was prepared comprising: 27.5% w/v unmodified pork skin gelatin having a molecular weight of about 100 kilodaltons, 4.1 M urea, 0.01 M phosphate buffered saline, and 0.76 mM tris(2,2′-bipyridyl) ruthenium (II) chloride hexa hydrate.

Second Part: A second flowable liquid composition was prepared as a 1 M aqueous solution of sodium persulfate.

### Example 2

### Kit Preparation for Two-Part Adhesive Composition

To make a kit for preparing a photocurable liquid adhesive, a 0.9 ml volume of the First Part composition of Example 1, sterilely prepared, is sterilely filled into the chamber of a first syringe barrel (1 ml volume capacity) equipped with a plunger, a cap is placed on the syringe tip, and the sterilely filled first syringe is sterilely sealed in a first medical package such as a gas impermeable foil medical package. A 0.1 ml volume of the Second Part composition of Example 1, sterilely prepared, is sterilely filled into the chamber of a second syringe barrel (1 ml volume capacity) equipped with a plunger (e.g. filled through the syringe tip opening), a cap if placed on the syringe tip, and the filled second syringe is sterilely sealed in a second medical package such as a gas impermeable foil medical package. The first and second medical packages containing respectively the first and second filled syringes are packaged together within in an outer package together with a sterilely packaged cannulated coupler for fluidly connecting the first and second syringes, to provide a kit for preparing a photocurable adhesive. The outer package further includes therein instructions for coupling the first and second syringes with the cannulated coupler and then for repeatedly and alternately depressing the plungers of the first and second syringes to mix the contents of the syringes.

### Example 3

### Preparation of Further Two-Part Adhesive Composition

First Part: A first flowable liquid composition was prepared comprising: 27.5% w/v unmodified pork skin gelatin having a molecular weight of about 100 kilodaltons, 4.1 M urea, 0.01 M phosphate buffered saline, and 0.76 mM tris(2,2′-bipyridyl) ruthenium (II) chloride hexahydrate.

Second Part: Sodium persulfate is provided as a dry powder.

### Example 4

### Further Kit Preparation for Two-Part Adhesive Composition

To make a kit for preparing a photocurable liquid adhesive, a 1 ml volume of the First Part composition of Example 3, sterilely prepared, is sterilely filled into the chamber of a first syringe barrel equipped with a plunger, a cap is placed on the syringe tip, and the filled syringe is sterilely sealed within a gas-impermeable foil package. A 0.24 gram amount of the dry powder sodium persulfate provided as the Second Part of Example 3 is filled into the chamber of a second syringe barrel equipped with a plunger, and the syringe barrel is equipped with a cap on the syringe tip. The second syringe is packaged within a Tyvek® (spunbound polyethylene fiber) film package and subjected to terminal sterilization with ethylene oxide gas to sterilize the dry powder sodium persulfate. The gas-impermeable foil package containing the sterile-filled first syringe and the terminally sterilized second syringe in the Tyvek film package are packaged together in an outer package (for example a box) along with a sterilely packaged cannulated coupler (having an internal static mixer) for fluidly connecting the respective tips of the first and second syringes, and also along with a sterilely packaged dispensing tip connectable to the tip of the first syringe and/or the tip of the second syringe, to provide a kit for preparing a photocurable adhesive. The outer package further includes therein printed material with instructions for use of the kit, the instructions including an instruction for coupling the first and second syringes with the cannulated coupler and mixing the contents of the syringes by repeatedly and alternately depressing the plungers of the first and second syringes.

### Example 5

### Phenol Enriched Gelatin Prepared with Bolton-Hunter Reagent

Nippi MediGelatin (derived from porcine skin; M_{w} approximately 100 kilodaltons) was dissolved in high purity water with 6.18 g/L boric acid, 9.54 g/L sodium borate, and 4.38 g/L sodium chloride at 30° C. and 60° C. at a concentration of 10 g/L in a 1 L reaction volume (n=1). 200 mL aliquots of this solution were extracted into 500 mL Erlenmeyer flasks and combined with 3.5 mL of a Bolton-Hunter reagent/DMSO solution (Bolton-Hunter reagent=N-succinimidyl-3-[4-hydroxyphenyl]propionate). The Bolton-Hunter/DMSO solution was prepared at a concentration such that the final concentration of Bolton-Hunter reagent in the gelatin solution ranged from 0.2 to 5 g/L. The mixture was reacted at 40° C. in a shaken incubator for two hours. The solution was dialyzed, dried, and analyzed for phenol content using absorbance at 280 nm. Unless noted otherwise, groups had a replicate size of n=3. Each group was evaluated for normality and compared across groups for equal variance. Groups were then compared for statistical difference using a one-way ANOVA (α=0.05) and Tukey post-hoc tests.

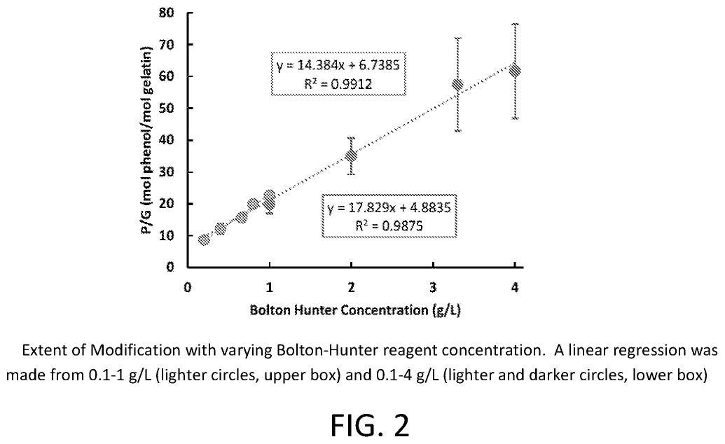

The results are summarized in the graph of FIG. 2. Phe/P values (mole phenol/mole gelatin—labeled more specifically as “P/G” in FIG. 2) for the modified materials ranged from just under 10 to about 60 (the unmodified gelatin had a Phe/P value of about 3). At all concentrations at and below 1 g/L Bolton-Hunter reagent, the resulting phenol enriched gelatin was soluble in PBS. The relationship between Bolton-Hunter reagent concentration and extent of phenol enrichment was generally linear in this work, with a 10% change in Bolton-Hunter concentration causing a ˜6% change in the molar ratio of phenol to gelatin. At Bolton-Hunter reagent concentrations at 2 g/L and higher, the relationship became less linear and the measurements had higher standard deviation. In addition, the modified gelatin became less soluble in PBS, with protein modified at 5 g/L becoming completely insoluble. These results indicate that a severe excess of Bolton-Hunter reagent and a resulting very high Phe/P value for the phenol enriched protein can decrease the solubility of the modified protein in an aqueous liquid.

### Example 6

### Photocurable Adhesives and Kits with Combinations of Gelatin and Phenol Enriched Gelatin

The procedures of Example 3 and 4 were repeated, except the First Part of the adhesive composition was prepared comprising 27.5% w/v of various mixtures of unmodified pork skin gelatin having a molecular weight of about 100 kilodaltons and phenol enriched pork skin gelatin prepared as described in Example 5 and having a Phe/P value of about 20, 4.1 M urea, 0.01 M phosphate buffered saline, and 0.76 mM tris(2,2′-bipyridyl) ruthenium (II) chloride hexahydrate. First, second and third mixtures included the unmodified gelatin and the phenol enriched gelatin in a ratio of 5:1, 1:1 and 1:5, respectively, to provide for relatively low, medium, and high reaction site density photocurable adhesives.

In additional work, lower and higher liquid volume adhesive kits were prepared using the above procedure of this Example 6. In lower volume products, 0.5 ml of the First Part sterile liquid preparation was filled into the first syringe and 0.12 grams of Second Part sodium persulfate powder was filled into the second syringe. In higher volume products, first and second syringes were used having a 3 ml volume capacity, and 3 ml of the First Part sterile liquid preparation was filed into the first syringe and 0.72 g of the Second Part sodium persulfate powder was filled into the second syringe.

### Example 7

### Phenol Enriched Gelatin Prepared by Carbodiimide Reaction

Gelatin was modified to include additional phenolic groups using EDC (1-ethyl-3-(3-dimethylaminopropyl)carbodiimide hydrochloride), NHS (N-hydroxysuccinimide), and HPPA (3-(4-Hydroxyphenyl)propionic acid). A precipitate was first prepared using a 5:2:1 ratio of NHS:HDC:HPPA at concentrations of 325 mM NHS, 130 mM EDC and 65 mM HPPA. First, HPPA was solubilized in a 0.1M MES, 0.9% Sodium Chloride, pH 4.7 buffer on a stir plate at 200 rpm. Once the HPPA was dissolved, the EDC and NHS were added to the solution. After 15-20 minutes a precipitate began to form. The solution was allowed to react for 2 to 4 hours and then vacuum filtered. Following double filtration of the solution, the precipitate captured on the filter paper was allowed to dry in a fume hood for at least 24 hours. Once the precipitate was dry, it was utilized as solubilized in DMSO in 4× mass in place of the Bolton-Hunter Reagent to add phenolic groups to the gelatin.

Modified gelatin prepared using the precipitate in place of Bolton-Hunter reagent was formulated into photocurable adhesive compositions using a phosphate buffered saline medium, bipyridyl) ruthenium (II) chloride hexahydrate and sodium persulfate. Photocurable adhesive formulations of having 5:1, 1:1 and 0:1 ratios of unmodified gelatin to modified gelatin were prepared. The thus prepared photocurable adhesives demonstrated the ability to cure under visible light.

The uses of the terms “a” and “an” and “the” and similar references herein (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the products or methods defined by the claims.

While embodiments of the disclosure have been illustrated and described in detail in the drawings and foregoing description, the same is to be considered as illustrative and not restrictive in character, it being understood that only some embodiments have been shown and described and that all changes and modifications that come within the spirit of the disclosures herein are desired to be protected.

1. A kit for preparing a photocurable liquid adhesive, comprising:
a first container defining a first chamber and containing a sterile liquid preparation in the first chamber, the sterile liquid preparation including an aqueous liquid, one or more polymers containing phenolic groups, and a photoactivatable metal ligand complex;
a second container defining a second chamber and containing an electron acceptor in the second chamber.

2. The kit of claim 1, wherein the first container and/or the second container is a syringe barrel.

3. The kit of claim 1, wherein the one or more polymers containing phenolic groups includes a natural polymer and/or a phenol enriched natural polymer.

4. The kit of claim 1, wherein the natural polymer is collagen, gelatin, or a collagen peptide composition having an average molecular weight of less than about 20 kilodaltons, and wherein the phenol enriched natural polymer is phenol enriched collagen, phenol enriched gelatin, or a phenol enriched collagen peptide composition having an average molecular weight of less than about 20 kilodaltons.

5. The kit of claim 1, wherein the one or more polymers containing phenolic groups includes a phenol enriched natural polymer having a Phe/P value of at least about 7.

6. The kit of claim 5, wherein the phenol enriched natural polymer includes added 4-hydroxyphenyl groups.

7. The kit of claim 1, wherein the electron acceptor in the second chamber is in dry powder form.

8. The kit of claim 7, wherein the sterile liquid preparation has a viscosity at 20° C. of greater than about 300 centipoise.

9. The kit of claim 1, wherein the one or more polymers containing phenolic groups is present in the sterile liquid preparation at a total concentration in the range of about 1 to about 40% weight/volume.

10. The kit of claim 1, wherein the one or more polymers containing phenolic groups is constituted of collagen, phenol enriched collagen, gelatin, phenol enriched gelatin, a collagen peptide composition having an average molecular weight of less than 20 kilodaltons, a phenol enriched collagen peptide composition having an average molecular weight of less than 20 kilodaltons, or a mixture of any two thereof or of any three or more thereof.

11. The kit of claim 1, wherein the one or more polymers containing phenolic groups is constituted of gelatin, phenol enriched gelatin, or a mixture thereof.

12. The kit of claim 11, wherein the one or more polymers is constituted of a mixture of gelatin and phenol enriched gelatin.

13. The kit of claim 12, wherein the phenol enriched gelatin includes added 4-hydroxyphenyl propionyl groups.

14. The kit of claim 1, wherein the electron acceptor is a persulfate compound, a periodate compound, a perbromate compound, or a perchlorate compound, vitamin B12, Co(III) (NH₃)SCI₂⁺, cerium (IV) sulphate dehydrate, ammonium cerium (IV) nitrate, oxalic acid or EDTA.

15. The kit of claim 14, wherein the electron acceptor is a persulfate compound, a periodate compound, a perbromate compound, or a perchlorate compound; and, preferably wherein the electron acceptor is sodium persulfate.

16. The kit of claim 1, also including means for shielding the sterile liquid preparation from light that would photoactivate the photoactivatable metal ligand complex; optionally, wherein the means for shielding is provided by the first container being impermeable to the light and/or by the first container being received in a package that is impermeable to the light.

17. The kit of claim 1, wherein the one or more polymers containing phenolic groups includes or is constituted of gelatin, phenol enriched gelatin, or a mixture thereof, and wherein the sterile liquid preparation further includes an agent for inhibiting thermoreversible gelling of the gelatin, the phenol enriched gelatin, or the mixture of gelatin and phenol enriched gelatin.

18. The kit of claim 17, wherein the agent is urea.

19. The kit of claim 1, wherein the one or more polymers containing phenolic groups includes or is constituted of a collagen peptide composition having an average molecular weight of less than about 20 kilodaltons, a phenol enriched collagen peptide composition having an average molecular weight of less than about 20 kilodaltons, or a mixture thereof.

20. The kit of claim 19, wherein the collagen peptide composition is a mixture of the collagen peptide composition and the phenol enriched collagen peptide composition.

21. The kit of claim 19, wherein the phenol enriched collagen peptide composition includes added 4-hydroxyphenyl groups, preferably added 4-hydroxyphenyl propionyl groups.

22-27. (canceled)

28. A liquid adhesive composition, comprising:
a photocurable liquid preparation including
(i) an aqueous liquid,
(ii) one or more polymers containing phenolic groups selected from collagen, phenol enriched collagen, gelatin, phenol enriched gelatin, a collagen peptide composition having an average molecular weight of less than 20 kilodaltons, and a phenol enriched collagen peptide composition having an average molecular weight of less than 20 kilodaltons,
(iii) a photoactivatable metal ligand complex, and
(iv) an electron acceptor, wherein the photocurable liquid preparation remains in a liquid state at 20° C.

29-43. (canceled)

44. A kit for preparing a photocurable adhesive according to claim 28, comprising:
a first container defining a first chamber and containing a sterile liquid preparation in the first chamber, the sterile liquid preparation including
(i) the aqueous liquid,
(ii) the one or more polymers containing phenolic groups, and
(iii) the photoactivatable metal ligand complex, wherein the sterile liquid preparation remains in a liquid state at 20° C.;
a second container defining a second chamber and containing the electron acceptor in the second chamber.

45-65. (canceled)

66. A method for preparing a photocurable liquid adhesive, comprising:
providing a kit according to claim 1; and
mixing the sterile liquid preparation and the electron acceptor.

67. A method for providing an adhesive on a surface, comprising:
applying a photocurable liquid adhesive of claim 28 to the surface; and
irradiating the photocurable liquid adhesive with visible light so as to cure the photocurable liquid adhesive.

68-75. (canceled)

76. A method for manufacturing a kit for preparing a photocurable liquid adhesive, comprising:
sterilely filling a sterile liquid preparation into a chamber defined in a first container, the sterile liquid preparation including an aqueous liquid, one or more polymers containing phenolic groups, and a photoactivatable metal ligand complex;
providing a second container defining a second chamber and containing an electron acceptor in sterile condition in the second chamber; and
packaging the first container and the second container together in a package.

77-78. (canceled)
